(19) United States
(12) Patent Application Publication
(10) Pub. No.: US 2023/0130285 A1
(43) Pub. Date: Apr. 27, 2023

(54) PERCUTANEOUS CIRCULATORY SUPPORT DEVICE FACILITATING THROMBI DISSOLUTION

(71) Applicant: Boston Scientific Scimed, Inc., Maple Grove, MN (US)

(72) Inventors: Anthony Malone, Galway (IE); Olena Pernatiy, Galway (IE); Javier Palomar-Moreno, Galway (IE)

(21) Appl. No.: 17/974,849

(22) Filed: Oct. 27, 2022

Related U.S. Application Data
(60) Provisional application No. 63/272,479, filed on Oct. 27, 2021.

(51) Int. Cl. A61M 60/148; A61M 60/216; A61M 60/422; A61M 60/419; A61M 60/825; A61M 60/414

(52) U.S. Cl. CPC A61M 60/148; A61M 60/216; A61M 60/414; A61M 60/419; A61M 60/422; A61M 60/825

(57) ABSTRACT

A percutaneous circulatory support device includes a housing and an impeller disposed within the housing. The impeller is rotatable relative to the housing to cause blood to flow through the percutaneous circulatory support device. The device further includes a reservoir disposed within the housing, and the reservoir carries at least one anticoagulant.

### CROSS REFERENCE TO RELATED APPLICATION

This application claims priority to Provisional Application No. 63/272,479 filed Oct. 27, 2021, which is herein incorporated by reference in its entirety.

### TECHNICAL FIELD

The present disclosure relates to percutaneous circulatory support systems. More specifically, the disclosure relates to percutaneous circulatory support devices that facilitate thrombi dissolution.

### BACKGROUND

Percutaneous circulatory support devices can provide transient support for up to approximately several weeks in patients with compromised heart function or cardiac output. Operation of such devices, however, may cause some amount of thrombosis (that is, formation of blood clots). Accordingly, there is a need for improved devices that facilitate thrombi dissolution.

### SUMMARY

In an Example 1, a percutaneous circulatory support device comprising a housing; an impeller disposed within the housing, the impeller being rotatable relative to the housing to cause blood to flow through the percutaneous circulatory support device; and a reservoir disposed within the housing, the reservoir carrying at least one anticoagulant.

In an Example 2, the percutaneous circulatory support device of Example 1, wherein the reservoir comprises a mesh coated with the at least one anticoagulant.

In an Example 3, the percutaneous circulatory support device of either of Examples 1 or 2, wherein the at least one anticoagulant comprises heparin.

In an Example 4, the percutaneous circulatory support device of any of Examples 1 to 3, further comprising a motor operatively coupled to the impeller, the motor rotating the impeller relative to the housing to cause blood to flow through the percutaneous circulatory support device.

In an Example 5, the percutaneous circulatory support device of Example 4, further comprising a driving magnet operatively coupled to the motor and a driven magnet operatively coupled to the driving magnet, the motor rotating the impeller, via the driving magnet and the driven magnet, relative to the housing to cause blood to flow through the percutaneous circulatory support device.

In an Example 6, the percutaneous circulatory support device of Example 5, wherein the reservoir is disposed between the impeller and the driven magnet.

In an Example 7, the percutaneous circulatory support device of either of Examples 5 or 6, further comprising an impeller shaft being fixed relative to the impeller and the driven magnet.

In an Example 8, the percutaneous circulatory support device of Example 7, wherein the impeller shaft extends through and rotates relative to the reservoir.

In an Example 9, the percutaneous circulatory support device of and of Examples 1 to 8, wherein the housing further comprises an outlet, and the outlet extends both proximally and distally beyond the reservoir.

In an Example 10, a percutaneous circulatory support device comprises a housing; an impeller disposed within the housing, the impeller being rotatable relative to the housing to cause blood to flow in a proximal direction and through the percutaneous circulatory support device; and a reservoir coupled to the housing and being disposed proximally relative to the impeller, the reservoir carrying at least one anticoagulant.

In an Example 11, the percutaneous circulatory support device of Example 10, wherein the reservoir comprises a mesh coated with the at least one anticoagulant.

In an Example 12, the percutaneous circulatory support device of either of Examples 10 or 11, further comprising a motor operatively coupled to the impeller, the motor rotating the impeller relative to the housing to cause blood to flow through the percutaneous circulatory support device.

In an Example 13, the percutaneous circulatory support device of Example 12, further comprising a driving magnet operatively coupled to the motor and a driven magnet operatively coupled to the driving magnet, the motor rotating the impeller, via the driving magnet and the driven magnet, relative to the housing to cause blood to flow through the percutaneous circulatory support device.

In an Example 14, the percutaneous circulatory support device of Example 13, wherein the reservoir is disposed between the impeller and the driven magnet.

In an Example 15, the percutaneous circulatory support device of either of Examples 13 or 14, further comprising an impeller shaft being fixed relative to the impeller and the driven magnet.

In an Example 16, a percutaneous circulatory support device comprises an impeller housing; an impeller disposed within the impeller housing, the impeller being rotatable relative to the impeller housing to cause blood to flow through the percutaneous circulatory support device; and a reservoir disposed within the impeller housing, the reservoir carrying at least one anticoagulant and being configured to release the at least one anticoagulant into blood flowing through the percutaneous circulatory support device.

In an Example 17, the percutaneous circulatory support device of Example 16, wherein the reservoir comprises a mesh coated with the at least one anticoagulant.

In an Example 18, the percutaneous circulatory support device of Example 16, wherein the at least one anticoagulant comprises heparin.

In an Example 19, the percutaneous circulatory support device of Example 16, further comprising a motor operatively coupled to the impeller, the motor rotating the impeller relative to the impeller housing to cause blood to flow through the percutaneous circulatory support device.

In an Example 20, the percutaneous circulatory support device of Example 19, further comprising a driving magnet operatively coupled to the motor and a driven magnet operatively coupled to the driving magnet, the motor rotating the impeller, via the driving magnet and the driven magnet, relative to the impeller housing to cause blood to flow through the percutaneous circulatory support device.

In an Example 21, the percutaneous circulatory support device of Example 20, wherein the reservoir is disposed between the impeller and the driven magnet.

In an Example 22, the percutaneous circulatory support device of Example 20, further comprising an impeller shaft being fixed relative to the impeller and the driven magnet.

In an Example 23, the percutaneous circulatory support device of Example 22, wherein the impeller shaft extends through and rotates relative to the reservoir.

In an Example 24, the percutaneous circulatory support device of Example 16, wherein the impeller housing further comprises an outlet, and the outlet extends both proximally and distally beyond the reservoir.

In an Example 25, a percutaneous circulatory support device comprises an impeller housing; an impeller disposed within the impeller housing, the impeller being rotatable relative to the impeller housing to cause blood to flow in a proximal direction and through the percutaneous circulatory support device; and a reservoir coupled to the impeller housing and being disposed proximally relative to the impeller, the reservoir carrying at least one anticoagulant and being configured to release the at least one anticoagulant into blood flowing through the percutaneous circulatory support device.

In an Example 26, the percutaneous circulatory support device of Example 25, wherein the reservoir comprises a mesh coated with the at least one anticoagulant.

In an Example 27, the percutaneous circulatory support device of Example 25, further comprising a motor operatively coupled to the impeller, the motor rotating the impeller relative to the impeller housing to cause blood to flow through the percutaneous circulatory support device.

In an Example 28, the percutaneous circulatory support device of Example 27, further comprising a driving magnet operatively coupled to the motor and a driven magnet operatively coupled to the driving magnet, the motor rotating the impeller, via the driving magnet and the driven magnet, relative to the impeller housing to cause blood to flow through the percutaneous circulatory support device.

In an Example 29, the percutaneous circulatory support device of Example 28, wherein the reservoir is disposed between the impeller and the driven magnet.

In an Example 30, the percutaneous circulatory support device of Example 28, further comprising an impeller shaft being fixed relative to the impeller and the driven magnet.

In an Example 31, the percutaneous circulatory support device of Example 30, wherein the impeller shaft extends through and rotates relative to the reservoir.

In an Example 32, a method for using a percutaneous circulatory support device comprises positioning the percutaneous circulatory support device at a target location within a patient; rotating an impeller of the percutaneous circulatory support device to cause blood to flow through the percutaneous circulatory support device; and releasing at least one anticoagulant from a reservoir of the percutaneous circulatory support device into blood flowing through the percutaneous circulatory support device.

In an Example 33, the method of Example 32, wherein the reservoir comprises a mesh coated with the at least one anticoagulant.

In an Example 34, the method of Example 32, wherein the at least one anticoagulant comprises heparin.

In an Example 35, the method of Example 32, wherein rotating the impeller comprises rotating the impeller relative to the reservoir.

While multiple embodiments are disclosed, still other embodiments of the present invention will become apparent to those skilled in the art from the following detailed description, which shows and describes illustrative embodiments of the invention. Accordingly, the drawings and detailed description are to be regarded as illustrative in nature and not restrictive.

### BRIEF DESCRIPTION OF THE DRAWINGS

FIG. 1 is a side sectional view of an illustrative mechanical circulatory support device (also referred to herein, interchangeably, as a “blood pump”), in accordance with embodiments of the subject matter disclosed herein.

FIG. 2 is a perspective view of the mechanical circulatory support device of FIG. 1, with housing components of the device shown in phantom lines, in accordance with embodiments of the subject matter disclosed herein.

FIG. 3 is a perspective view of several internal components of the mechanical circulatory support device of FIG. 1, including a driven magnet, an impeller assembly, and an anticoagulant reservoir, in accordance with embodiments of the subject matter disclosed herein.

FIG. 4 is a perspective view of the anticoagulant reservoir of FIG. 3, in accordance with embodiments of the subject matter disclosed herein.

FIG. 5 is a perspective view of another illustrative anticoagulant reservoir, in accordance with embodiments of the subject matter disclosed herein.

While the invention is amenable to various modifications and alternative forms, specific embodiments have been shown by way of example in the drawings and are described in detail below. The intention, however, is not to limit the invention to the particular embodiments described. On the contrary, the invention is intended to cover all modifications, equivalents, and alternatives falling within the scope of the invention as defined by the appended claims.

### DETAILED DESCRIPTION

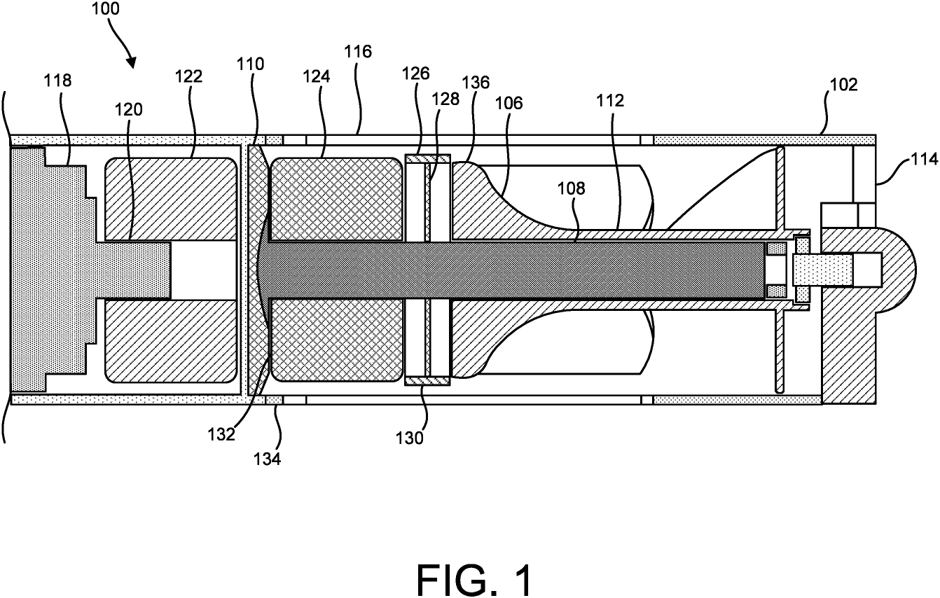

FIG. 1 depicts a partial side sectional view of an illustrative mechanical circulatory support device 100 (also referred to herein, interchangeably, as a “blood pump”) in accordance with embodiments of the subject matter disclosed herein. The device 100 may form part of a percutaneous circulatory support system, together with a guidewire and an introducer sheath (not shown). More specifically, the guidewire and the introducer sheath may facilitate percutaneously delivering the device 100 to a target location within a patient, such as within the patient’s heart. Alternatively, the device 100 may be delivered to a different target location within a patient.

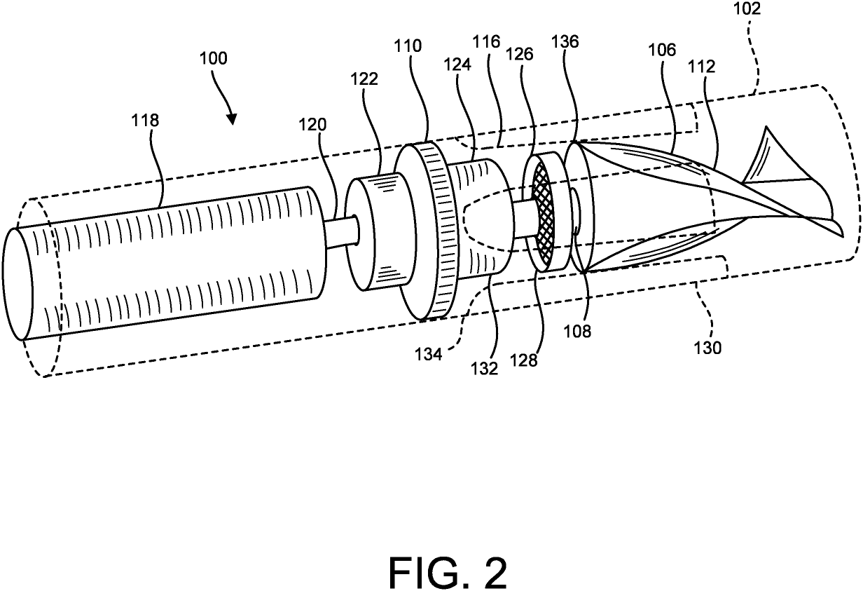

With continued reference to FIG. 1 and additional reference to FIG. 2, the device 100 generally includes an impeller housing 102 and a motor housing 104. In some embodiments, the impeller housing 102 and the motor housing 104 may be integrally or monolithically constructed. In other embodiments, the impeller housing 102 and the motor housing 104 may be separate components configured to be removably or permanently coupled.

The impeller housing 102 carries an impeller assembly 106 therein. The impeller assembly 106 includes an impeller shaft 108 that is rotatably supported by at least one bearing, such as a bearing 110. The impeller assembly 106 also includes an impeller 112 that rotates relative to the impeller housing 102 to drive blood through the device 100. More specifically, the impeller 112 causes blood to flow from a blood inlet 114 (FIG. 1) formed on the impeller housing 102, through the impeller housing 102, and out of a blood outlet 116 formed on the impeller housing 102. In some embodiments and as illustrated, the impeller shaft 108 and the impeller 112 may be separate components, and in other embodiments the impeller shaft 108 and the impeller 112 may be integrated. In some embodiment and as illustrated, the inlet 114 and/or the outlet 116 may each include multiple apertures. In other embodiments, the inlet 114 and/or the outlet 116 may each include a single aperture. In some embodiments and as illustrated, the inlet 114 may be formed on an end portion of the impeller housing 102 and the outlet 116 may be formed on a side portion of the impeller housing 102. In other embodiments, the inlet 114 and/or the outlet 116 may be formed on other portions of the impeller housing 102. In some embodiments, the impeller housing 102 may couple to a distally extending cannula (not shown), and the cannula may receive and deliver blood to the inlet 114.

With continued reference to FIGS. 1 and 2, the motor housing 104 carries a motor 118, and the motor 118 is configured to rotatably drive the impeller 112 relative to the impeller housing 102. In the illustrated embodiment, the motor 118 rotates a drive shaft 120, which is coupled to a driving magnet 122. Rotation of the driving magnet 122 causes rotation of a driven magnet 124, which is connected to and rotates together with the impeller assembly 106. More specifically, in embodiments incorporating the impeller shaft 108, the impeller shaft 108 and the impeller 112 are configured to rotate with the driven magnet 124. In other embodiments, the motor 118 may couple to the impeller assembly 106 via other components.

In some embodiments, a controller (not shown) may be operably coupled to the motor 118 and configured to control the motor 118. In some embodiments, the controller may be disposed within the motor housing 104. In other embodiments, the controller may be disposed outside of the motor housing 104 (for example, in a catheter handle, an independent housing, etc.). In some embodiments, the controller may include multiple components, one or more of which may be disposed within the motor housing 104. According to embodiments, the controller may be, may include, or may be included in one or more Field Programmable Gate Arrays (FPGAs), one or more Programmable Logic Devices (PLDs), one or more Complex PLDs (CPLDs), one or more custom Application Specific Integrated Circuits (ASICs), one or more dedicated processors (e.g., microprocessors), one or more Central Processing Units (CPUs), software, hardware, firmware, or any combination of these and/or other components. Although the controller is referred to herein in the singular, the controller may be implemented in multiple instances, distributed across multiple computing devices, instantiated within multiple virtual machines, and/or the like. In other embodiments, the motor 118 may be controlled in other manners.

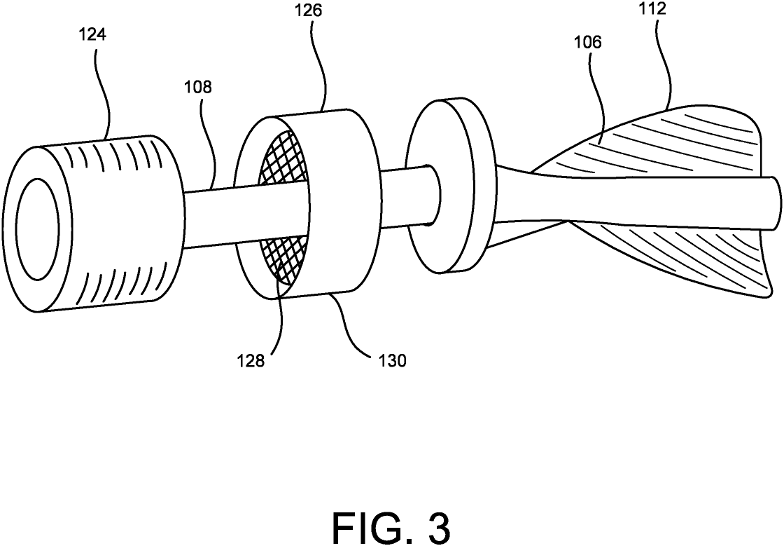

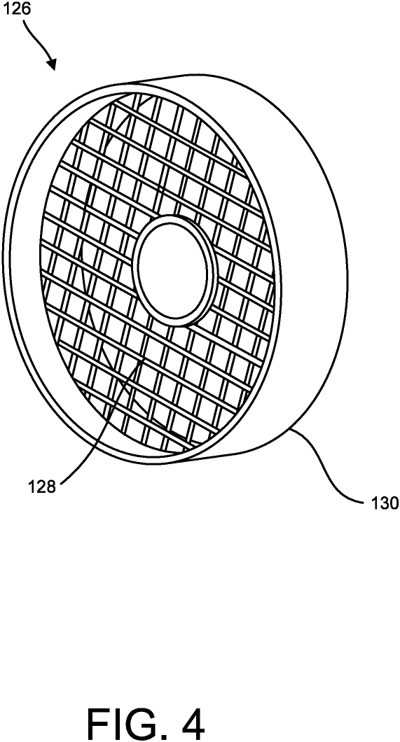

With further reference to FIGS. 1 and 2 and additional reference to FIGS. 3 and 4, the device 100 facilitates thrombi dissolution. More specifically, the device 100 includes a reservoir 126 that contains and releases into the blood located within and/or flowing through the device 100 one or more pharmaceutical agents or drugs that inhibit blood coagulation (referred to herein simply as “anticoagulants”). The anticoagulants may include, for example, heparin, dalteparin, or enoxaparin. In some embodiments, the anticoagulants may be accompanied by one or more solid materials that carry and then release the anticoagulants in a controlled manner as they degrade upon exposure to blood, such as polylactic glycolic acid (PLGA). In some embodiments and as illustrated, the reservoir 126 may include a thin filter or mesh 128 that surrounds the impeller shaft 108, and an outer cylinder 130 that surrounds the mesh 128. The mesh 128 and/or the cylinder 130 may be coated with the anticoagulants. In other embodiments and as shown elsewhere, the reservoir 126 may alternatively take different forms. In some embodiments and as illustrated, the reservoir 126 is fixed relative to the impeller housing 102, and the impeller shaft 108 extends through and rotates relative to the reservoir 126. In some embodiments and as illustrated, the reservoir 126 is disposed proximally from the impeller 112, more specifically between the impeller 112 and the driven magnet 124.

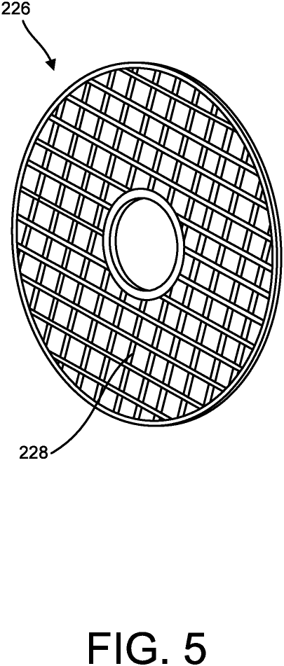

Anticoagulant reservoirs of circulatory support devices in accordance with embodiments of the subject matter disclosed herein may take other forms. FIG. 5 illustrates an example of such a reservoir 226, which may be used as part of the device 100 instead of the reservoir 126. The reservoir 226 includes a thin filter or mesh 228 that is coated with the coagulants. However, and unlike the reservoir 126, the reservoir 226 lacks an outer cylinder.

Referring again to FIGS. 1 and 2, the device 100 may also include one or more features that facilitate reduced device-induced hemolysis compared to conventional devices. For example, the apertures of the outlet 116 may be relatively long compared to those of conventional devices. More specifically, the apertures of the outlet 116 may each extend to a proximal end 132 of the driven magnet 124. Such apertures inhibit blood from pooling in the proximal end portion 134 of the impeller housing 102, which reduces hemolysis and/or thrombosis. As another example, a proximal end portion 136 of the impeller 112 may have a flattened shape, in contrast to lips or peaks of the impellers of conventional devices. Such a shape inhibits blood from forming and pooling in vortices adjacent to the impeller 112.

In some embodiments, other or additional components of the device 100 are coated with and release the anticoagulants. For example, the driven magnet 124, the impeller 112, and/or the inner surface of the impeller housing 102 may be coated with and release the anticoagulants. In some embodiments, the anticoagulants may be accompanied by one or more solid materials that carry and then release the anticoagulants in a controlled manner as they degrade upon exposure to blood, such as polylactic glycolic acid (PLGA)

Various modifications and additions can be made to the exemplary embodiments discussed without departing from the scope of the present invention. For example, while the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

We claim:

1. A percutaneous circulatory support device, comprising:
an impeller housing;
an impeller disposed within the impeller housing, the impeller being rotatable relative to the impeller housing to cause blood to flow through the percutaneous circulatory support device; and
a reservoir disposed within the impeller housing, the reservoir carrying at least one anticoagulant and being configured to release the at least one anticoagulant into blood flowing through the percutaneous circulatory support device.

2. The percutaneous circulatory support device of claim 1, wherein the reservoir comprises a mesh coated with the at least one anticoagulant.

3. The percutaneous circulatory support device of claim 1, wherein the at least one anticoagulant comprises heparin.

4. The percutaneous circulatory support device of claim 1, further comprising a motor operatively coupled to the impeller, the motor rotating the impeller relative to the impeller housing to cause blood to flow through the percutaneous circulatory support device.

5. The percutaneous circulatory support device of claim 4, further comprising a driving magnet operatively coupled to the motor and a driven magnet operatively coupled to the driving magnet, the motor rotating the impeller, via the driving magnet and the driven magnet, relative to the impeller housing to cause blood to flow through the percutaneous circulatory support device.

6. The percutaneous circulatory support device of claim 5, wherein the reservoir is disposed between the impeller and the driven magnet.

7. The percutaneous circulatory support device of claim 5, further comprising an impeller shaft being fixed relative to the impeller and the driven magnet.

8. The percutaneous circulatory support device of claim 7, wherein the impeller shaft extends through and rotates relative to the reservoir.

9. The percutaneous circulatory support device of claim 1, wherein the impeller housing further comprises an outlet, and the outlet extends both proximally and distally beyond the reservoir.

10. A percutaneous circulatory support device, comprising:
an impeller housing;
an impeller disposed within the impeller housing, the impeller being rotatable relative to the impeller housing to cause blood to flow in a proximal direction and through the percutaneous circulatory support device; and
a reservoir coupled to the impeller housing and being disposed proximally relative to the impeller, the reservoir carrying at least one anticoagulant and being configured to release the at least one anticoagulant into blood flowing through the percutaneous circulatory support device.

11. The percutaneous circulatory support device of claim 10, wherein the reservoir comprises a mesh coated with the at least one anticoagulant.

12. The percutaneous circulatory support device of claim 10, further comprising a motor operatively coupled to the impeller, the motor rotating the impeller relative to the impeller housing to cause blood to flow through the percutaneous circulatory support device.

13. The percutaneous circulatory support device of claim 12, further comprising a driving magnet operatively coupled to the motor and a driven magnet operatively coupled to the driving magnet, the motor rotating the impeller, via the driving magnet and the driven magnet, relative to the impeller housing to cause blood to flow through the percutaneous circulatory support device.

14. The percutaneous circulatory support device of claim 13, wherein the reservoir is disposed between the impeller and the driven magnet.

15. The percutaneous circulatory support device of claim 13, further comprising an impeller shaft being fixed relative to the impeller and the driven magnet.

16. The percutaneous circulatory support device of claim 15, wherein the impeller shaft extends through and rotates relative to the reservoir.

17. A method for using a percutaneous circulatory support device, comprising:
positioning the percutaneous circulatory support device at a target location within a patient;
rotating an impeller of the percutaneous circulatory support device to cause blood to flow through the percutaneous circulatory support device; and
releasing at least one anticoagulant from a reservoir of the percutaneous circulatory support device into blood flowing through the percutaneous circulatory support device.

18. The method of claim 17, wherein the reservoir comprises a mesh coated with the at least one anticoagulant.

19. The method of claim 17, wherein the at least one anticoagulant comprises heparin.

20. The method of claim 17, wherein rotating the impeller comprises rotating the impeller relative to the reservoir.
